(19) United States
(12) Patent Application Publication
(10) Pub. No.: US 2012/0115798 A1
(43) Pub. Date: May 10, 2012

(54) METHOD FOR IMPROVING THE IMMUNITY OF A COMPANION ANIMAL

(76) Inventors: Stefan Patrick Massimino, Kettering, OH (US); Gary Mitchell Davenport, Dayton, OH (US); Jin Zhang, Clayton, OH (US)

(21) Appl. No.: 12/939,594

(22) Filed: Nov. 4, 2010

(51) Int. Cl. A61K 31/70; A61P 37/04

(52) U.S. Cl. 514/23

(57) ABSTRACT

A method of improving the immunity of a companion animal. The method can include administering to the companion animal a glucose anti-metabolite. The glucose anti-metabolite can be 2-deoxy-D-glucose; 5-thio-D-glucose; 3-O-methylglucose; 1,5-anhydro-D-glucitol; 2,5-anhydro-D-glucitol; 2,5-anhydro-D-mannitol; mannoheptulose; and mixtures and combinations thereof. The companion animal can be a dog or a cat. Improving the immunity can include altering the proliferative ability of T and B immune cells, and altering the relative distribution of immune cell phenotypes, for example.

### FIELD

Embodiments of the invention relate to a method of improving the immunity of a companion animal. More particularly, but not exclusively, embodiments of the invention relate to a method of administering to a companion animal a glucose anti-metabolite to improve the immunity of the companion animal.

### BACKGROUND

Biological theories have correctly predicted the finding that a restriction of caloric intake by food deprivation slows down certain undesirable cellular processes in laboratory animals, many associated with aging and age-related diseases.

In particular, caloric restriction has been shown to consistently extend the life span, delay onset and slow tumor progression, and retard physiologic aging in many systems. Indeed, research spanning more than seventy years has shown that caloric restriction is a nutritional intervention that consistently extends longevity in animals. See Weindruch and Walford, “The Retardation of Aging and Disease by Dietary Restriction,” Springfield, Ill.: Charles C. Thomas (1988); Yu, “Modulation of Aging Processes by Dietary Restriction,” Boca Raton: CRC Press (1994); and Fishbein, “Biological Effects of Dietary Restriction,” Springer, New York (1991). These effects of caloric restriction on life span and tumorigenesis have been reported numerous times since the early studies of McKay. See McKay et al., “The Effect of Retarded Growth Upon the Length of Lifespan and Upon Ultimate Body Size,” J. Nutr., Vol. 10, pp. 63-79 (1935). Indeed, over the past two decades, a resurgence of interest in caloric restriction in gerontology has led to the general acceptance that this dietary manipulation slows physiologic aging in many systems. See Weindruch and Walford, “The Retardation of Aging and Disease by Dietary Restriction,” Springfield, Ill.: Charles C. Thomas (1988); Yu, “Modulation of Aging Processes by Dietary Restriction,” Boca Raton: CRC Press (1994); and Fishbein, “Biological Effects of Dietary Restriction,” Springer, New York (1991) and Masoro, E. J. “Overview of Caloric Restriction and Ageing,” Mech. Aging Dev., Vol. 126, pp 913-922 (2005).

Reductions in fasting glucose and insulin levels and improvements in insulin sensitivity are readily measured biomarkers of caloric restriction. Calorically restricted rodents exhibit lower fasting glucose and insulin levels, and the peak glucose and insulin levels reached during a glucose challenge are reduced in those on caloric restriction. See Kalant et al., “Effect of Diet Restriction on Glucose Metabolism and Insulin Responsiveness and Aging Rats,” Mech. Aging Dev., Vol. 46, pp. 89-104 (1988). It is also known that hyperinsulinemia is a risk factor associated with several such disease processes, including heart disease and diabetes (Balkau and Eschwege, Diabetes Obes. Metab. 1 (Suppl. 1): S23-31, 1999). Reduced insulin levels and body temperature are two of the most reliable indicators of this altered metabolic profile (Masoro et al., J. Gerontol. Biol. Sci. 47:B202-B208, 1992); Koizumi et al., J. Nutr. 117: 361-367, 1987; Lane et al., Proc. Nat. Acad. Sci. 93:4154-4164, 1996).

Glucose anti-metabolites such as 2-deoxy-D-glucose are compounds related to glucose. However, due to structural differences from glucose such compounds block or inhibit certain aspects of carbohydrate metabolism and may therefore mimic the effects of caloric restriction (Rezek et al., J. Nutr. 106:143-157, 1972). These anti-metabolites exert a number of physiological effects, including reduction of body weight, decrease in plasma insulin levels, reduction of body temperature, retardation of tumor formation and growth, and elevation of circulating glucocorticoid hormone concentrations. (For a review see Roth et al., Ann. NY Acad. Sci. 928:305-315, 2001). These physiological effects result from inhibition of carbohydrate metabolism.

As such, use of glucose anti-metabolites as components for improving the immunity in mammals would be beneficial. The interaction between nutrition and the immune response has been an area of intense research over the past five decades. A bi-directional interaction between nutrition, immune response, and infectious disease was suggested by Scrimshaw (Schrimshaw N S, Taylor C E, Gordon J E. Interaction of nutrition and infection. Am J Med Sci 1959; 237:367-403) in the 1950's. Subsequently, it was recognized that malnourished individuals were at risk for infection. Follow up studies have demonstrated that deficiencies of most micronutrients result in impaired host defense. On the other hand, others have demonstrated the supplementation of certain nutrients beyond accepted requirements may improve certain indices of the immune response. Taken together, it is quite evident that nutrition is able to impact immunity. The dysregulation in immune function is a well-documented consequence of aging. This dysregulation can lead to an increased incidence of morbidity (illness) and mortality (death). Cell-mediated immunity, primarily T cells is clearly the component of the immunity most adversely affected with advancing age. For review, see Pawelec (Pawelec G, Wagner W, Adibzadeh M, Engel A. T cell immunosenescence in vitro and in vivo. Exp Gerontol 1999; 34:419-29). Age-related T cell immunity dysfunction has been implicated as the cause of many chronic degenerative diseases in elderly humans, including arthritis, cancer, autoimmune diseases, and increased susceptibility to infectious diseases. Many theories exist that have been put forth to try and explain the mechanism(s) responsible for this decline, but no one theory can fully account for all the changes observed. Senior dogs have been reported to show a decreased immunity response compared to younger dogs. Older dogs also differ in the makeup of their immunity compared to younger dogs. Based on these observations, the aging process results in a dysregulation of the immune response in dogs too, as is similar with other species and further provides an opportunity for nutritional modulation in this, or related species.

Thus, it would be beneficial to provide nutrition such as glucose anti-metabolites having physiological effects on the immunity, such as by altering the proliferative ability of T and B immune cells, and altering the relative distribution of immune cell phenotypes, for example. It would also be beneficial to provide a composition comprising such glucose anti-metabolite components. Accordingly, embodiments of the invention relate to such benefits.

### SUMMARY

Embodiments herein relate to a method for improving immunity of a companion animal. The method can include, in one embodiment, administering to the companion animal a glucose anti-metabolite in an amount effective to improve the immunity of the companion animal. The glucose anti-metabolite can be selected from the group consisting of 2-deoxy-D-glucose; 5-thio-D-glucose; 3-O-methylglucose; 1,5-anhydro-D-glucitol; 2,5-anhydro-D-glucitol; 2,5-anhydro-D-mannitol; mannoheptulose; and mixtures and combinations thereof. The companion animal can be a canine and a feline. In specific embodiments, the glucose anti-metabolite can be mannoheptulose. In specific embodiments, the composition can be a kibble, which can be nutritionally balanced and which can contain less than about 5% mannoheptulose.

The method can include feeding from about 1 mg/kg to about 50 mg/kg mannoheptulose to the companion animal per day. The method can also include feeding from about 1 mg to about 1000 mg mannoheptulose per day.

In certain embodiments, the method for improving immunity of a companion animal includes administering to the companion animal a glucose anti-metabolite in an amount effective to improve the immunity of the companion animal, wherein improving the immunity includes improving the ability of the immune system of the companion animal to respond such that the proliferative ability of T and B immune cells to respond to a stimulation challenge is altered after administration of the glucose anti-metabolite.

In certain embodiments, the method for improving immunity of a companion animal includes administering to the companion animal a glucose anti-metabolite in an amount effective to improve the immunity of the companion animal, wherein improving the immunity comprises attenuating the decline of the immune system by attenuating the age associated increase in CD18+ cells.

### BRIEF DESCRIPTION OF THE DRAWINGS

FIG. 1 is a graph of CD18+ immune cells for dog group 1.

FIG. 2 is a graph of CD18+ immune cells for dog group 2.

FIG. 3 is a graph of T cell mitogenic stimulation to ConA for dog group 1.

FIG. 4 is a graph of T cell mitogenic stimulation to ConA for dog group 2.

FIG. 5 is a graph of T cell mitogenic stimulation to PHA for dog group 1.

FIG. 6 is a graph of T cell mitogenic stimulation to PHA for dog group 2.

FIG. 7 is a graph of B cell mitogenic stimulation to PWM for dog group 1.

FIG. 8 is a graph of B cell mitogenic stimulation to PWM for dog group 2.

FIG. 9 is a graph of the average dog group 1 serum 8-OHdG level.

### DETAILED DESCRIPTION

### Definitions

As used herein, the articles including “the”, “a”, and “an”, when used in a claim or in the specification, are understood to mean one or more of what is claimed or described.

As used herein, the terms “include”, “includes”, and “including” are meant to be non-limiting.

As used herein, the term “plurality” means more than one.

As used herein, the terms “animal” or “pet” mean a domestic animal including, but not limited to domestic dogs (canines), cats (feline), horses, cows, ferrets, rabbits, pigs, rats, mice, gerbils, hamsters, horses, and the like. Domestic dogs and domestic cats are particular examples of pets and are referred to herein as “companion animals.” It should be understood that throughout this disclosure when using the term animal, pet, or companion animal, the animal, pet, or companion animal is in a non-diseased state, unless otherwise stated.

As used herein, the terms “animal feed”, “animal feed compositions”, “animal feed kibble”, “pet food”, or “pet food composition” all mean a composition intended for ingestion by a pet. Pet foods can include, without limitation, nutritionally balanced compositions suitable for daily feed, as well as supplements and/or treats, which may or may not be nutritionally balanced.

As used herein, the term “nutritionally balanced” means that a composition, such as pet food, has known required nutrients to sustain life in proper amounts and proportions based on recommendations of recognized authorities, including governmental agencies, such as, but not limited to, Unites States Food and Drug Administration's Center for Veterinarian Medicine, the American Feed Control Officials Incorporated, in the field of pet nutrition, except for the additional need for water.

All oral doses of the invention are calculated per kilogram of body weight of the mammal unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” . . . list of items . . . “and combinations and mixtures thereof”

Referenced herein are trade names for components including various ingredients utilized in embodiments of the invention. The inventors herein do not intend to be limited by materials under a certain trade name. Equivalent materials (e.g., those obtained from a different source under a different name or reference number) to those referenced by trade name may be substituted and utilized in the descriptions herein.

The processes, methods, compositions, and apparatuses herein may comprise, consist essentially of, or consist of any of the features or embodiments as described herein.

In the description of the various embodiments of the disclosure, various embodiments or individual features are disclosed. As will be apparent to the ordinarily skilled practitioner, all combinations of such embodiments and features are possible and can result in preferred executions of the disclosure. While various embodiments and individual features of the invention have been illustrated and described, various other changes and modifications can be made without departing from the spirit and scope of the invention. As will also be apparent, all combinations of the embodiments and features taught in the foregoing disclosure are possible and can result in preferred executions of the invention.

### EMBODIMENTS OF THE INVENTION

Embodiments of the invention relate to compositions comprising a glucose anti-metabolite component selected from the group consisting of 2-deoxy-D-glucose; 5-thio-D-glucose; 3-O-methylglucose; 1,5-anhydro-D-glucitol; 2,5-anhydro-D-glucitol; 2,5-anhydro-D-mannitol; mannoheptulose; and mixtures and combinations thereof. Without intending to be limited by theory, these components are accepted to be glucose anti-metabolites. In another embodiment, the components may be present in the recited compositions by virtue of a component of plant matter such as avocado, or other enriched source of mannoheptulose such as alfalfa, fig, primrose, and the like.

Embodiments of the invention also relate to a method of improving the immunity of mammals. Such methods relate to administering to the mammal a composition as disclosed herein, wherein the composition is effective at improving the immunity of the mammal regardless of the age of the mammal. In one specific embodiment, the method relates to improving the ability of the immune system to respond, also termed improving immune response, of a mammal, such as a companion animal, by administration of a composition comprising a glucose anti-metabolite. In one other specific embodiment, the method relates to maintaining and/or attenuating a decline of the immune system with aging of a mammal, such as a companion animal, by administration of a composition comprising a glucose anti-metabolite.

Immunity can be divided into innate and adaptive immunity. The adaptive branch of the immune system is represented by cellular and humoral immunity and can be defined by improved T and B cell responses, which can be measured by assays such as, but not limited to, tritiated thymidine lymphoproliferative response. The innate branch of the immune system is represented by CD18+ immune cells, which can be measured by assays such as, but not limited to, altered relative and absolute percent of white blood cell populations as measured by immunofluorescence.

Additionally, improving the immunity can include altering the proliferative ability of T and B immune cells, and altering the relative distribution of immune cell phenotype, for example.

The mammals disclosed herein can include vertebrates and invertebrates, such as for example insects (e.g., the fruit fly) and/or nematodes (e.g., Caenorbabditis elegans). Humans and companion animals are disclosed herein.

The glucose anti-metabolite components as disclosed herein include 2-deoxy-D-glucose, 5-thio-D-glucose, 3-O-methylglucose, anhydrosugars including 1,5-anhydro-D-glucitol, 2,5-anhydro-D-glucitol, and 2,5-anhydro-D-mannitol, mannoheptulose, and mixtures and combinations thereof. Mannoheptulose is one particular glucose anti-metabolite. In one embodiment, mannoheptulose may be present in the recited compositions as a component of plant matter such as an avocado, avocado extract, avocado meal, avocado concentrate or other enriched source of mannoheptulose. Non-limiting examples of enriched sources of mannoheptulose include alfalfa, fig, or primrose. The plant matter may include the fruit, seed (or pit), branches, leaves, or any other portion of the relevant plant or combinations thereof.

Avocado (also commonly referred to as alligator pear, aguacate, or palta) contains unusually enriched sources of mannoheptulose, as well as related sugars and other carbohydrates. Avocado is a sub-tropical evergreen tree fruit, growing most successfully in areas of California, Florida, Hawaii, Guatemala, Mexico, the West Indies, South Africa, and Asia.

Species of avocado include, for example, Persea Americana and Persea nubigena, including all cultivars within these illustrative species. Cultivars may include ‘Anaheim,’ ‘Bacon,’ ‘Creamhart,’ ‘Duke,’ ‘Fuerte,’ ‘Ganter,’ ‘Gwen,’ ‘Hass,’ ‘Jim,’ ‘Lula,’ ‘Lyon,’ ‘Mexicola Grande,’ ‘Murrieta Green,’ ‘Nabal,’ ‘Pinkerton,’ ‘Queen,’ ‘Puebla,’ ‘Reed,’ ‘Rincon,’ ‘Ryan,’ ‘Spinks,’ ‘Topa Topa,’ ‘Whitsell,’ ‘Wurtz,’ and ‘Zutano.’ The fruit of the avocado is particularly preferred for use herein, which may contain the pit or wherein the pit is removed or at least partially removed. Fruit from Persea Americana is particularly preferred for use herein, as well as fruit from cultivars which produce larger fruits (e.g., about 12 ounces or more when the fruit is mature), such as Anaheim, Creamhart, Fuerte, Hass, Lula, Lyon, Murrieta Green, Nabal, Queen, Puebla, Reed, Ryan and Spinks.

Plant matter from alfalfa, fig, or primrose is also reported to provide relatively high levels of mannoheptulose. Alfalfa is also referred to as Medicago sativa. Fig or Ficus carica (including Cluster fig or Sycamore fig, for example) may also be used, as well as primrose or Primula officinalis.

It has been discovered that particular levels of a component selected from 2-deoxy-D-glucose; 5-thio-D-glucose; 3-O-methylglucose; 1,5-anhydro-D-glucitol; 2,5-anhydro-D-glucitol; 2,5-anhydro-D-mannitol; mannoheptulose; and mixtures and combinations thereof can be useful herein. In particular, it has been found that relatively low levels, as well as relatively high doses of the component, while useful, may provide less than optimal efficacy for desired purposes. Dosage will depend upon the glucose anti-metabolite component used and will vary depending upon the size and condition of the mammal to which the glucose anti-metabolite is to be administered. Dosage in the range of about 0.0001 or about 0.001 grams/kg to about 1 g/kg can be beneficial in some embodiments. As used herein, when dosage in mg/kg is used, the “mg” refers to the level of the component, such as mannoheptulose, and “kg” refers to kilograms of body weight of the mammal, such as a dog or cat. Dosage at the lower range may also be appropriate when using 2-deoxy-D-glucose in large animals. Higher dosage, particularly of compounds such as 5-thio-D-glucose or mannitol, may also be readily tolerated. In one embodiment, the dosage of the component provided to a mammal on a daily basis may be from about 0.1, 0.5, 1, 2, or 5 mg/kg to about 15, 20, 50, 100, 150, or 200 mg/kg, and all combinations of these ranges, wherein “mg” refers to the level of the component and “kg” refers to kilograms of body weight of the mammal. In one embodiment, the dosage to the mammal, on a daily basis, may be from about 1 mg/kg to about 15 mg/kg, from about 2 mg/kg to about 10 mg/kg, or from about 2 mg/kg to about 5 mg/kg. In one embodiment, the dosage to the mammal, on a daily basis, may be from about 1 mg/kg to about 5 mg/kg, from about 1.5 mg/kg to about 5 mg/kg, from about 2 mg/kg to about 5 mg/kg, or about 2 mg/kg. In certain embodiments, these amounts may translate to compositions comprising less than about 5%, or less than about 2%, or from about 0.0001% to about 0.5%, or from about 0.1% to about 10%, or from about 0.1% to about 5%, of the component, all by weight of the composition. All ranges therebetween are envisioned. The level of component may be determined by one of ordinary skill in the art based on a variety of factors, for example, the form of the composition (e.g., whether a dry composition, semi-moist composition, wet composition, or supplement, or any other form or mixture thereof). The ordinarily skilled artisan will be able to utilize the preferred dosage and determine the optimal level of component within a given composition.

Similarly, the overall dosage amount of the component on a daily basis provided to the mammal may be provided. Such a daily dosage amount can be from about 0.1 mg per day to about 1000 mg per day. Such daily dosage amounts can be dependent on the size of the mammal consuming the composition. For example, in one embodiment, larger mammals may consume more than smaller mammals. Of course, that is consistent with the dosing disclosed herein with respect to a dosing amount per mass of the mammal. Thus, in one embodiment, as the mammal increases in size, more of the composition can be administered.

Accordingly, in one embodiment, such a daily dosage amount can correspond to the dosage on a daily basis per mass of the mammal, as described herein. Specifically, daily dosage amounts can range, in some embodiments, from about 0.1 mg per day to about 1000 mg per day, or even more, depending on the size of the mammal and the daily dosage amounts as described above. In other embodiments, the daily dosage can be from about 1 mg per day to about 500 mg per day, or from about 1 mg per day to about 200 mg per day, or from about 1 mg per day to about 100 mg per day, or from about 5 mg day per day to about 100 mg per day, or from about 5 mg per day to about 80 mg per day, or from about 10 mg per day to about 50 mg per day, or about 40 mg per day. All ranges therebetween are also envisioned.

Similarly, wherein an extract or meal of plant matter is utilized in the compositions herein, levels of extract or meal may be dependent upon level of efficacious component within such extract or meal. Extracts and/or meals have been found herein which comprise from about 0.5% to about 99% of the glucose anti-metabolite component, alternatively from about 0.5% to about 75% of the glucose anti-metabolite component, alternatively from about 0.5% to about 50% of the glucose anti-metabolite component, alternatively, from about 0.5% to about 25% of the glucose anti-metabolite component, all by weight of the extract or meal. Extracts and/or meals have been found herein in which the glucose anti-metabolite component may be from about 0.5, 1, 2, 5, or 10% to about 15, 25, 50 or 75% by weight of the extract and/or meal.

Accordingly, embodiments of the invention are directed to a composition that is intended for ingestion by a mammal Compositions include foods intended to supply necessary dietary requirements, as well as treats (e.g., biscuits) or other food supplements. Optionally, the composition herein may be a dry composition (for example, kibble), semi-moist composition, wet composition, or any mixture thereof. Alternatively or additionally, the composition is a supplement, such as a gravy, drinking water, yogurt, powder, suspension, chew, treat (e.g., biscuits) or any other delivery form.

Moreover, in one embodiment the composition can be nutritionally balanced, such as a pet food kibble. In another embodiment, the composition is not nutritionally balanced, such as a supplement, treat, or other delivery form for a pet.

The compositions used herein may optionally comprise one or more further components. Other components are beneficial for inclusion in the compositions used herein, but are optional for purposes of the invention. In one embodiment, the compositions may comprise, on a dry matter basis, from about 10% to about 90% crude protein, alternatively from about 20% to about 50% crude protein, alternatively from about 20% to about 40% crude protein, by weight of the composition, or alternatively from about 20% to about 35% crude protein, by weight of the composition. The crude protein material may comprise vegetable-based proteins such as soybean, cereals (corn, wheat, etc), cottonseed, and peanut, or animal-based proteins such as casein, albumin, and meat protein. Non-limiting examples of meat protein useful herein include a protein source selected from the group consisting of beef, pork, lamb, poultry, fish, and mixtures thereof.

Furthermore, the compositions may comprise, on a dry matter basis, from about 5% to about 40% fat, alternatively from about 10% to about 35% fat, by weight of the composition.

Embodiments related to compositions of the invention may further comprise a source of carbohydrate. In one embodiment, the compositions may comprise from about 35%, by weight of the composition, up to about 50%, by weight of the composition, carbohydrate source. In other embodiments, the composition can comprise from about 35% to about 45%, by weight of the composition, or from about 40% to 50%, by weight of the composition, carbohydrate source. Grains or cereals such as rice, corn, milo, sorghum, barley, wheat, and the like are illustrative sources of carbohydrate.

The compositions may also contain other materials such as, but not limited to, dried whey and other dairy by-products, beet pulp, cellulose, fiber, fish oil, flax, vitamins, minerals, flavors, antioxidants, and taurine.

The compositions may also contain other optional ingredients. Optional ingredients can include Probiotic components (Bifidobacteria and/or Lactobacillus) and Prebiotic (fructooligosaccharides) components. Examples and amounts of Probiotic components and Prebiotic components that can be included are disclosed in United States Publication No. 2005/0158294, for example. Other optional ingredients that can be included are omega 6 and omega 3 fatty acids, carnitine, hexametaphosphate, glucosamine, chondroitin sulfate, carotenoids including beta carotene, vitamin E, and lutein, and those ingredients as shown in Table 1 below.

### Immunity

As disclosed herein, the compositions can be useful for improving the immunity of mammals, particularly companion animals. With respect to immunity, CD18 is a pan-leukocyte cell surface marker that has been shown to increase with age and during inflammation (Valente. Immunologic function in the elderly after injury—the neutrophil and innate immunity; J Trauma. 2009; 67(5):968-74). Leukocytes play an important role in the pathogenesis of tissue injury due to inflammation. At the site of increased leukocyte—endothelial cell interaction, leukocytes in the microcirculation interact with adhesion molecules on the endothelial cells, which lead to rolling, adhesion, and migration. The leukocytes subsequently release cytokines and produce proteases and superoxide radical species, which also participate in the inflammation cascade. It is these reactions of leukocytes that cause inflammatory tissue and endothelial cell injury. The expression of CD11b/CD18 (CR3), which is related to the phagocytic production of reactive oxygen intermediates (ROI) as described above, is up-regulated in monocytes from older subjects, in comparison to the younger subjects. As well, autoimmunity is due in part to the heterodimeric β2 integrin lymphocyte function-associated antigen-1 (LFA-1) CD11a/CD18 over expression. As a result, the increase in CD11a/CD18 antigen density on lymphocytes has been discussed as an event in the mechanism leading to the decreased lymphocyte proliferative response in vitro and to other immunological dysfunctions reported in old subjects (Chiricolo M, Morini M C, Mancini R, Beltrandi E, Belletti D, Conte R. Cell adhesion molecules CD11a and CD18 in blood monocytes in old age and the consequences for immunological dysfunction. Gerontology. 1995; 41(4):227-34). As a result of all these previously reported negative effects of CD18 over-expression seen with advancing age, it would be desirable to mitigate/attenuate the age-associated increase of CD18, which glucose anti-metabolites, and in particular mannoheptulose, are shown to do herein in companion animals, such as dogs, as provided for herein.

As shown in FIG. 1, when analyzed by age, dogs that were fed a test diet formulated to deliver 2 milligrams mannoheptulose per kilogram body weight of the animal showed a significant attenuation in the increase of CD18+ immune cells when compared against their diet matched controls. The increase in CD18+ immune cells is thought to be an event in the mechanism leading to the decreased lymphocyte proliferative response in vitro and to other immunological dysfunctions reported in older subjects, therefore an attenuation of this increase would be considered beneficial immunologically to the mammal.

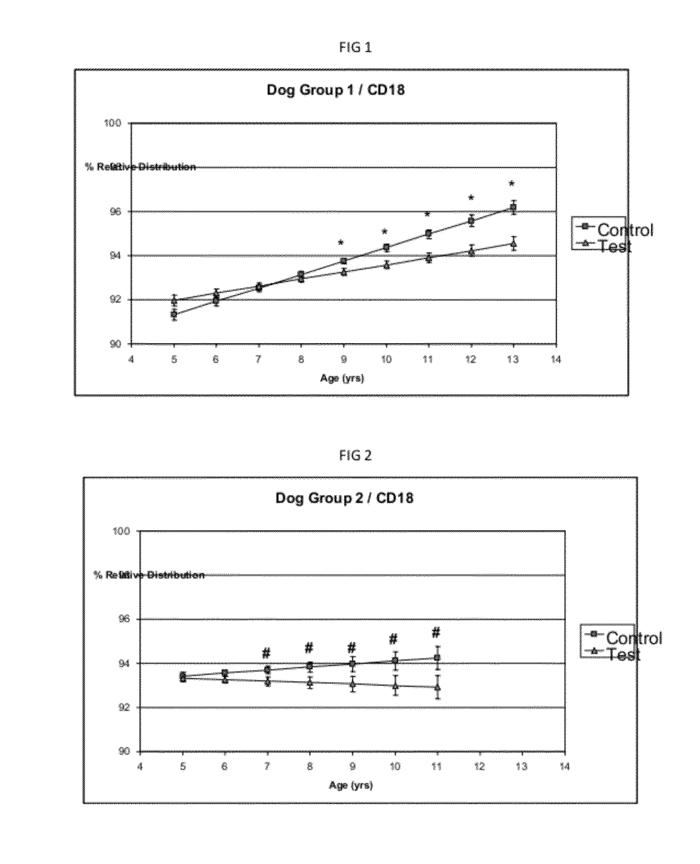

As shown in FIG. 2, when analyzed by age, dogs that were fed the test diet with mannoheptulose showed a trend for attenuation in the increase of CD18+ immune cells when compared against their diet matched controls. The increase in CD18+ immune cells is thought to be an event in the mechanism leading to the decreased lymphocyte proliferative response in vitro and to other immunological dysfunctions reported in older subjects, therefore an attenuation of this increase would be considered beneficial immunologically to the mammal.

The dogs of FIG. 1, associated with a dog group 1, represent an older group of dogs than those of FIG. 2, associated with a dog group 2, by about two years.

Concanavalin A (ConA) exhibits mitogenic activity, specifically with T-lymphocytes (Ruscetti and Chervenick 1975; Novogrodsky and Katchalski 1971; Perlmann et al. 1970). Phytohaemagglutinin (PHA), the lectin extract from the red kidney bean (Phaseolus vulgaris), contains potent, T-cell mitogenic activity (Hammerstrom, S. et al. (1982) Proc. Nall. Acad. Sci. USA 79, 1611-1615). Pokeweed mitogen (PWM), derived from Phytolacca Americana, is a B cell mitogen (Schreck et al., Annals of Clinical and Laboratory Science, Vol 12, Issue 6, 455-462). Together these three mitogens are used to stimulate immune cells in vitro in the lymphocyte blastogenesis assay, a well accepted indicator of immune response in vivo. Many studies have shown this assay to be responsive to the age-associated decline in immune system response (Goldrosen et al., 1977—Journal of surgical oncology 9: 229-234). A lower proliferative capacity indicates the immune cells cannot become activated and multiply as efficiently (a key step in the process of mounting an effective immune response). This decreased proliferative capacity has been attributed to the increased morbidity and mortality seen with advancing age and hence is an important indicator of immune capacity.

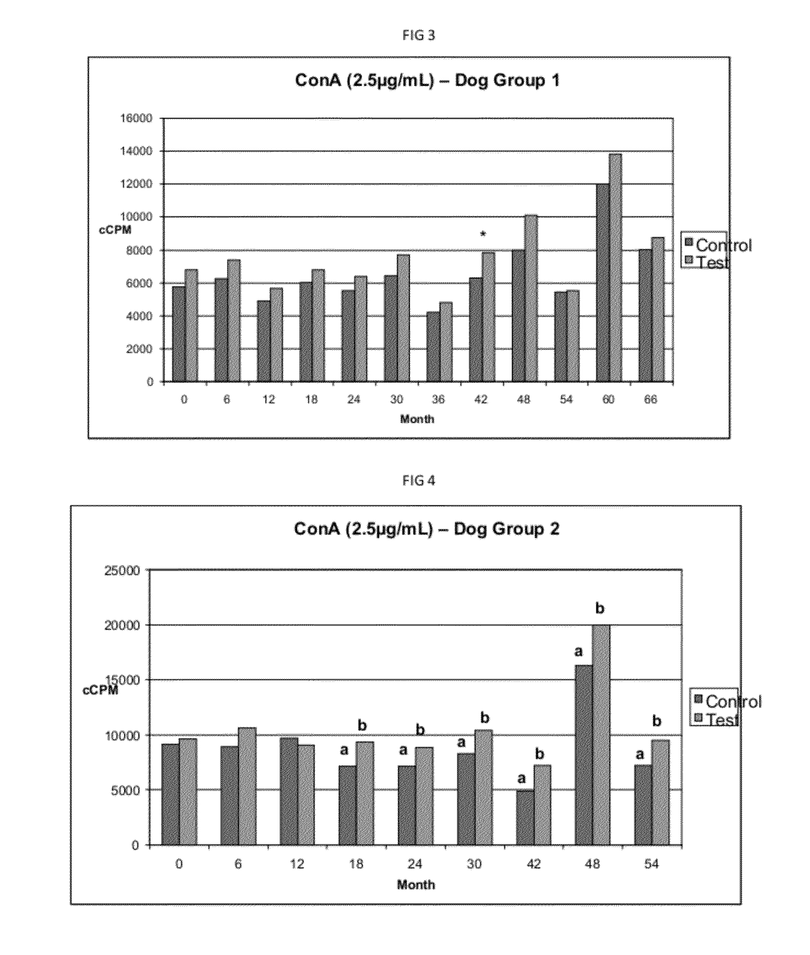

Data from ConA, PHA, and PWM is shown in FIGS. 3-8 for the two groups of dogs.

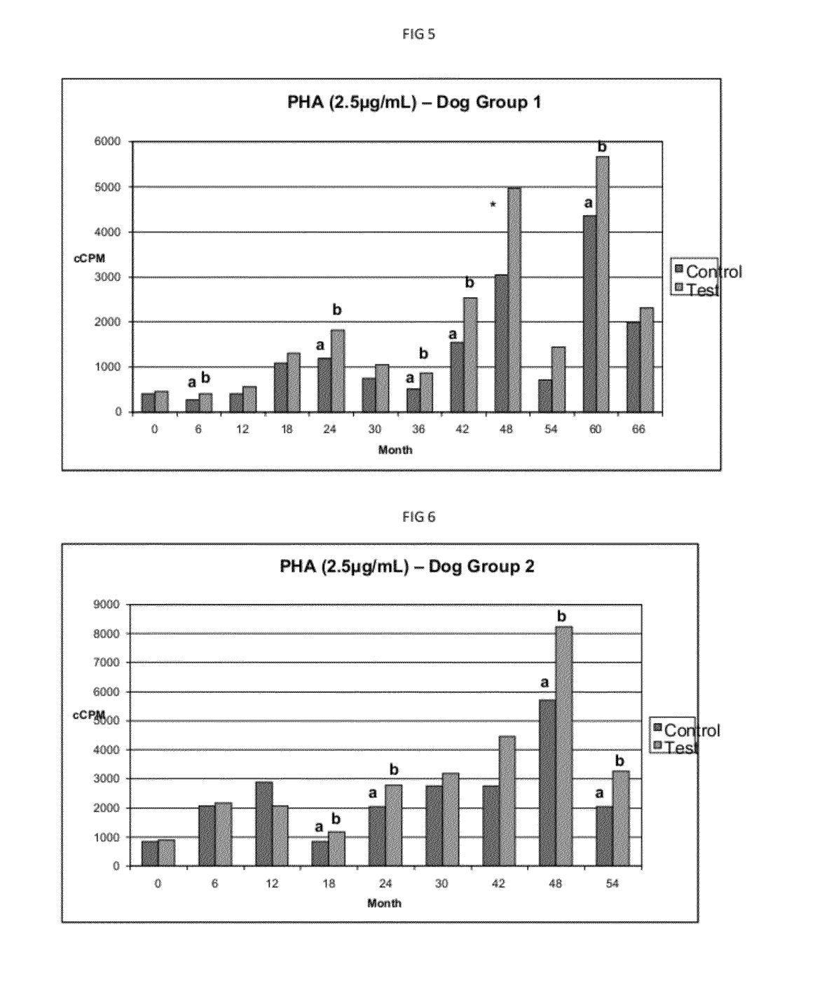

For ConA, FIGS. 3 and 4 indicate a benefit of mannoheptulose on T cell proliferative capability. All baseline values are similar (p>0.05), and either numerically, statistically trending, or statistically higher for dogs fed mannoheptulose-containing diets versus a control diet. Taken together, these observations support the conclusion that mannoheptulose is beneficial on this compartment of the immune cell population. T cell responsiveness declines with age and is a major contributing factor to the increased morbidity and mortality seen with advancing age. Therefore, reversing or mitigating this effect positively affects the health and immune response in dogs. Thus, FIGS. 3 and 4 show that dogs which are fed a test diet containing mannoheptulose have either a statistical trend or a statistical improvement in T cell mitogenic stimulation to ConA. This increase indicates that the T cells of dogs fed a mannoheptulose containing diet are better able to respond to a challenge when compared to T cells from dogs fed a control diet.

Similar to ConA, PHA responses shown in FIGS. 5 and 6 indicate a benefit of mannoheptulose on the ability of the T cell compartment to respond to mitogenic stimulation. All baseline values are similar (p>0.05), and during the treatment period, all PHA-stimulated T cell data are either numerically, statistically trending, or statistically higher for mannoheptulose fed dogs versus controls. Taken together with the evidence seen with ConA, these observations support the conclusion that mannoheptulose is beneficial on the T cell compartment of the immune cell population. It is well known that T cell responsiveness declines with age and is a major contributing factor to the increased morbidity and mortality seen with advancing age. Therefore, reversing or mitigating this effect positively affects the immune response, health, and wellness of dogs. FIGS. 5 and 6 above show equally that dogs which are fed a test diet containing mannoheptulose have either a statistical trend or a statistical improvement in T cell mitogenic stimulation to PHA. This increase indicates that the T cells of dogs fed a mannoheptulose containing diet are better able to respond to a challenge when compared to T cells from dogs fed a control diet.

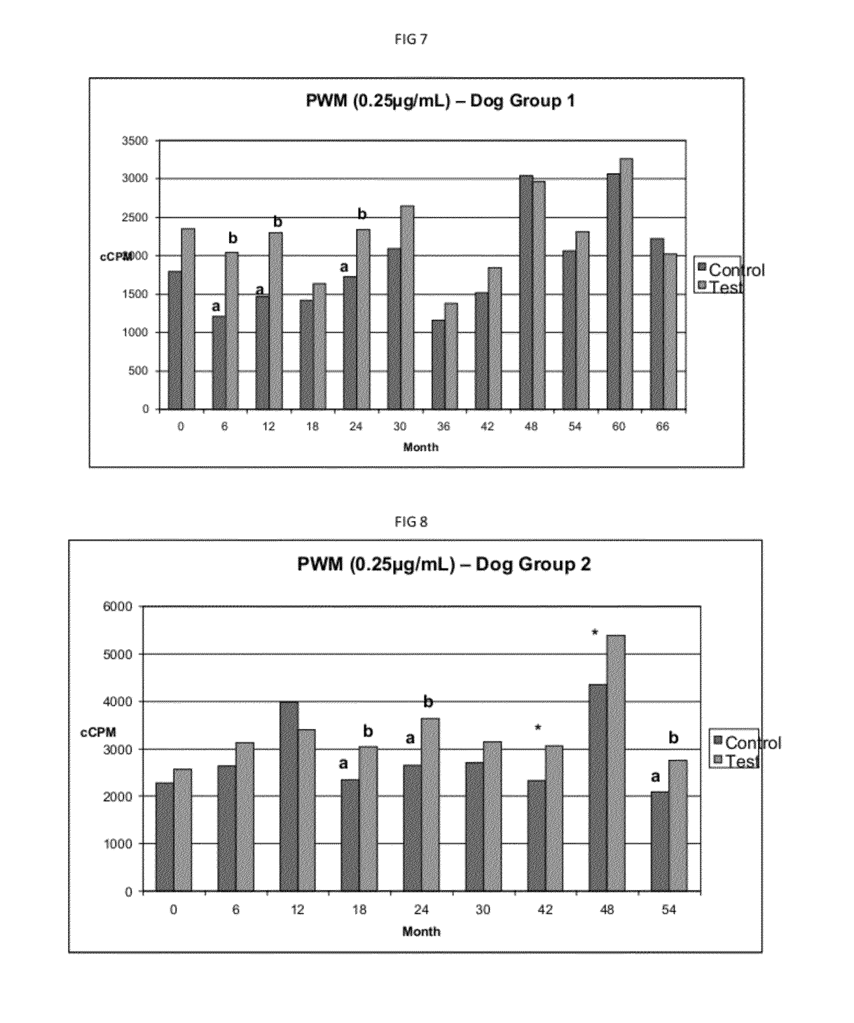

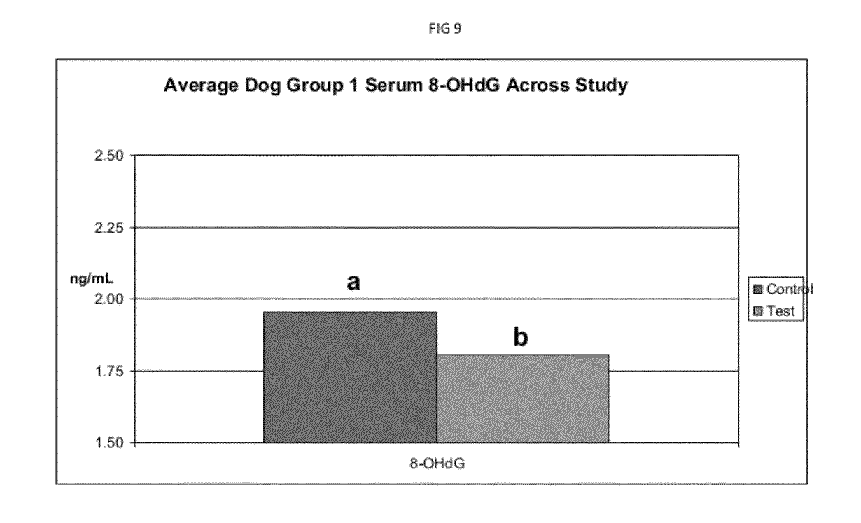

The results shown in FIGS. 7 and 8 indicate a benefit of feeding mannoheptulose on the B cell proliferative ability in dogs, shown as PWM in the figures. All baseline data are statistically similar and whenever a significant beneficial effect is seen, it is as a result of feeding mannoheptulose. B cell responsiveness is also known to decrease with age and is also another contributing factor to the increased morbidity and mortality seen with advancing age. Therefore, reversing or mitigating this effect positively affects the immune response, health, and wellness of dogs. FIGS. 7 & 8 above show equally that dogs which are fed a test diet containing mannoheptulose have either a statistical trend or a statistical improvement in B cell mitogenic stimulation to PWM. This increase indicates that the B cells of dogs fed a mannoheptulose containing diet are better able to respond to a challenge when compared to B cells from dogs fed a control diet.

8-Oxo-2′-deoxyguanosine (8-OHdG) is an oxidized derivative of deoxyguanosine. 8-OHdG is one of the major products of DNA oxidation. As part of the immune response, T and B cells must proliferate (become activated and divide). A key step to robust proliferation is DNA replication. If cells have damaged DNA or must repair DNA, this crucial step can be hindered. As shown in FIG. 9, dogs that were fed a test diet containing mannoheptulose had significantly lower serum 8-OHdG compared to dogs fed a control diet. By lowering DNA damage in the mannoheptulose fed dogs, immune cell replication would be facilitated, helping to enable a key step in the activation cascade of the immune system.

As a summary, Table 1 is provided below. Table 1 shows the specific response in the first column. The second column is titled “General aging” and indicates what happens to the marker as aging occurs in many species. The third column relates to dogs and matches the second column of general aging of many species and thus indicates that without mannoheptulose, the signs of general aging in dogs will continue. However, in the fourth column, the markers are shown for aging when dogs consume a diet comprising mannoheptulose. As can be seen, the T cell proliferation and B cell proliferation responses increase when dogs consume a diet comprising mannoheptulose, while T cell proliferation and B cell proliferation responses would normally decrease with age. The same is true for CD18+ cells and DNA damage as the consumption of a diet comprising mannoheptulose will decrease these markers in contract to their typical increase with aging. Thus, a diet comprising mannoheptulose can positively impact the immune response, especially by way of attenuating the decline in the immune response as the dog ages and by improving the ability of the immune system to respond when challenged.

TABLE 1

| | General aging | Without MH | With MH |
|---|---|---|---|
| T cell proliferation | ↓ | ↓ | ↑ |
| B cell proliferation | ↓ | ↓ | ↑ |
| % CD18 + cells | ↑ | ↑ | ↓ |
| DNA damage | ↑ | ↑ | ↓ |

In another embodiment, an article of commerce is provided. The article of commerce can include a package. Any standard packaging that is used for deliver and sale of the compositions as disclosed herein can be used. The package can contain compositions disclosed herein, such as compositions comprising mannoheptulose, or any other glucose anti-metabolite. The compositions can be nutritionally balanced pet food compositions. The package can include specific benefit statements written on the package. The specific benefit statements can relate to benefits that are provided to the consumer of the composition. The specific benefit statements can relate to improving the immunity when the composition is consumed. For example, the specific benefit statements can relate to improving the ability of the immune system of a companion animal to respond if the composition is administered to the companion animal. Another example includes specific benefit statements that can relate to maintaining and/or attenuating a decline of the immune system of an aging companion animal if the composition is administered to the aging companion animal. Thus, an article of commerce is provided, wherein the article of commerce comprises a package that contains a companion animal composition, wherein the package includes a benefit statement relating to improving the immunity of the companion animal if the companion animal consumes the companion animal composition contained within the package.

### EXAMPLES

The following examples are provided to illustrate embodiments of the invention and are not intended to limit the scope thereof in any manner

### Preparation of Mannoheptulose-containing Avocado Meal:

Fresh avocados (Lula variety) were obtained from Fresh King Incorporated (Homestead, Fla.). The avocados were manually split open and the pits were removed and discarded. The remaining skin and pulp were ground through a Hobart Commercial Food Preparation machine (Ser. No. 11-10410235) using a 12¼ sieve. The ground avocado was then transferred to an Edwards Freeze Drier (Super Modulyo Model, Crawely, Sussex, England). The freeze drier was set at −20° C. for the first 24 hours, −5° C. for the following 24 hours and 5° C. for the final 72 hours. Upon removal from the freeze drier, the meal was ground to a powder using a Straub Grinding Mill (model 4E, Philadelphia, Pa.). The avocado meal was analyzed and found to contain about 10.35% mannoheptulose, by weight of the meal. It should be noted that the amount of mannoheptulose found in avocados varies with the particular strain and state of ripeness.

### Preparation of Avocado Extract

Avocado extract containing enhanced levels of mannoheptulose is prepared in accordance with the following optional process, and utilized in compositions of embodiments of the invention.

Whole avocado fruit (about 900 kilograms) is provided. The fruit is split and the pits are removed, either partially or wholly, providing about 225 kilograms of pitted avocado halves. The raw avocado is charged to a disintegrator, whereupon some agitation, water (about 3000 kilograms) and CELLUBRIX (commercially available from Novozymes A/S) (about 1 liter) is further charged. The mixture is further agitated and concurrently heated to about 66° C. Upon completion of the charge, further CELLUBRIX (about 1 liter) is added, and the entire mixture is held under agitation for about 12 hours at a controlled pH of about 5.5. The temperature is then further increased to about 80° C. and then held for at least about 2 hours. The resulting digested plant mixture is then filtered at 80° C. to provide the carbohydrate extract as the filtrate. The carbohydrate extract is then evaporated in a simplified recirculation system at 80° C., under vacuum, to provide the carbohydrate extract having from about 10% to about 20% solids and a pH of about 5.5. The extract is then further concentrated using a refractance window dryer to provide about 100 kilograms of the extract as a crystalline or powder (a yield of about 11% carbohydrate extract, based on the starting mass of the whole avocado fruit, which is analyzed as a yield from about 0.25% to about 4.5% mannoheptulose, based on the starting mass of the whole avocado fruit). It should be noted the amount of mannoheptulose found in avocados varies with the particular strain and state of ripeness of the fruit. The extract may be used in the compositions of embodiments of the invention.

### Kibble Compositions

Table 1 illustrates two kibble compositions having the following components at the approximate indicated amounts are prepared using methods which are standard in the art, including extrusion, and are fed to dogs and/or cats as a daily feed:

TABLE 1

| | Component Amount | Component Amount |
|---|---|---|
| Component | indicated as Wt % | indicated as Wt % |
| Extract of Avocado* | 0.02 | 0.01 |
| Chicken, Chicken By- | 44 | 47 |
| product Meal, Fish Meal, | | |
| and Egg | | |
| Chicken Fat | 8 | 6 |
| Beet Pulp | 2 | 3 |
| Salts | 2.5 | 2 |
| Vitamins and Minerals** | 1 | 1 |
| Minors*** | 3.5 | 4 |
| Grains | Remainder | Remainder |
| (corn, sorghum, barley, | | |
| rice, wheat) | | |

*Avocado may be substituted with other plant matter having enhanced mannoheptulose content. The incorporation of a mannoheptulose source likely replaces a similar amount of a grain source in the composition.
**Vitamins and Minerals may include: Vitamin E, beta-carotene, Vitamin A, Ascorbic Acid, Calcium Pantothenate, Biotin, Vitamin B₁₂, Vitamin B₁, Niacin, Vitamin B₂, Vitamin B₆, Vitamin D₃, Vitamin D₂, Folic Acid, Choline Chloride, Inositol, Calcium Carbonate, Dicalcium Phosphate, Potassium Chloride, Sodium Chloride, Zinc Oxide, Manganese Sulfate, Copper Sulfate, Manganous Oxide, Ferrous Sulfate, Potassium Iodide, Cobalt Carbonate.
***Minors may include: Fish oil, flax seed, flax meal, cellulose, flavors, antioxidants, taurine, yeast, carnitine, chondroitin sulfate, glucosamine, lutein, rosemary extract.

### Administration

Eighty (n=80) Labrador Retrievers were randomized by age, gender, and littermate to receive either a complete and nutritionally balanced control diet that is similar to Eukanuba Senior Large Breed or an experimental diet that is identical to the control diet except for the inclusion of mannoheptulose as disclosed below. The dogs were split into two study groups.

Study 1: A total of 39 older Labrador Retrievers are fed a nutritionally-balanced composition providing mannoheptulose at levels of 0 or about 2 mg/kg of body weight of the dog. Average age of the dogs (12 neutered males, 27 spayed females) at the start of a 4-year study is 6.7 years with a range of 5.1 to 8.2 years of age for the youngest and oldest dog within the cohort, respectively. The control composition is fed as a nutritionally-balanced composition, and it contains no mannoheptulose (0 mg/kg), avocado extract, avocado meal, or avocado concentrate. The test composition is the nutritionally-balanced control composition formulated with avocado extract, avocado meal, or avocado concentrate to provide mannoheptulose at a dose of about 2 mg/kg body weight of the dog. Older dogs are fed one-half their daily allotment of food at 0730 and 1430 each day. Dogs were fed to maintain body weight and body composition score (BCS) within a 2-4 score range. If food adjustments were made, they were made on a quarterly basis. All dogs were fasted overnight and morning meals were withheld until blood collections could be conducted for all immune measurements. Water is provided ad lib.

Study 2: A total of 41 younger Labrador Retrievers are fed a nutritionally-balanced composition providing mannoheptulose at levels of 0 or about 2 mg/kg of body weight of the dog. Average age of the dogs (12 neutered males, 29 spayed females) at the start of the 36-month feeding study is 4.0 years with a range of 2.0 to 6.1 years of age for the youngest and oldest dog within the cohort, respectively. The control composition is fed as a nutritionally-balanced composition (Eukanuba Senior Maintenance Formula), and it contains no mannoheptulose (0 mg/kg), avocado extract, avocado meal, or avocado concentrate. The test composition is the nutritionally-balanced control composition formulated with avocado extract, avocado meal, or avocado concentrate to provide mannoheptulose at a dose of about 2 mg/kg body weight of the dog. Younger dogs are fed one-half their daily allotment of food at 0730 and 1430 each day. Dogs were fed to maintain body weight and body composition score (BCS) within a 2-4 score range. If food adjustments were made, they were made on a quarterly basis. However, all dogs were fasted overnight and morning meals were withheld until blood collections could be conducted for all immune measurements. Water is provided ad lib.

### Methods

### Blastogenesis Materials and Methods:

Canine whole blood was collected into heparin tubes and centrifuged at 600 g for 10 minutes. The buffy coat was transferred to a new sterile polypropylene tube and diluted to 13 ml with PBS. This blood mixture was then layered onto 9 ml of room temperature histopaque 1.077 and centrifuged for 30 minutes at 500 g. The PBMC layer was removed from the gradient and washed with PBS. Remaining red blood cells were removed with ACK lyses buffer (NH4CL-155 mM, EDTA-0.1 mM, KHCO3-10 mM, pH7.4) and PBMCs were washed again with PBS. Cell count was determined on a Z2 Coulter Counter (Beckman Coulter).

Mitogens were purchased from Sigma and diluted in complete media (RPMI with 10% FBS and 1% PenStrep). Diluted Mitogens were added to the wells of a 96 well tissue culture plate at the following concentrations: ConcavalinA 2.5 ug/ml, 5 ug/ml and 10 ug/ml. PHA 2.5 ug/ml, 10 ug/ml and 20 ug/ml. Pokeweed mitogen 0.25 ug/ml, 1 ug/ml and 5 ug/ml. PBMCs in complete media were also added to each well at 2×10̂5 cells per well. Total volume of each well is 200 ul. Plates were then incubated at 37 degrees in 5% CO2 for a total of 72 hrs. At 52 hrs (+/−2 hrs) the cells were pulsed with 1 uCi per well of 3H-thymidine. Cells were harvested and radioactivity counted by liquid scintillation. 8-OHDG Assay:

Assay was run using canine serum samples separated from whole blood. Amount of 8-OHDG is quantified using a commercially available ELISA kit from Oxis Health Products, catalog #21026.

Bring all reagents and samples to room temperature before use.

1. Reconstitute the Primary Antibody with the Primary Antibody Dilution Buffer.

2. Add 50 mL of sample or standard per well. To prevent edge effects, do not use outermost rows (Rows A and H).

3. Add 50 mL of reconstituted Primary Antibody to all wells except Blank. Seal plate tightly with Plate Seal. Shake plate from side to side to mix fully. Incubate at 37° C. for 1 hour.

4. Pour off contents of plate. Pipette 250 mL diluted Washing Buffer into each well. Wash thoroughly by agitation, dispose of Washing Buffer. Invert plate and blot against clean paper towel to remove any remaining washing buffer. Repeat wash twice.

5. Reconstitute the Secondary Antibody with the Secondary Antibody Dilution Buffer.

6. Add 100 mL of reconstituted Secondary Antibody per well. Seal plate tightly with Plate Seal. Shake plate from side to side to mix fully. Incubate at 37° C. for 1 hour.

7. Dilute the Chromogen with 100 volumes of Chromogen Dilution Buffer.

8. Repeat step 4.

9. Add 100 mL of the diluted Chromogen per well. Shake plate from side to side to mix fully. Incubate at room temperature in the dark for 15 minutes.

10. Add 100 mL of the Stop Solution, mix, wait 3 minutes and read the absorbance at 450 nm.

### Flow Cytometry—CD18 Method:

Canine whole blood was collected into EDTA tubes and centrifuged at 400 g for 30 minutes. The buffy coat was transferred to a new sterile polypropylene tube and diluted with 2 ml PBS. Red blood cells were lysed by adding 5 ml H2O for one minute and stopping with 5 ml 2X cold PBS. Cells were centrifuged at 300 g for 10 min to pellet. The lysis step was repeated until a white cell pellet was obtained. Cells were then resuspended in 0.8 ml FACS Wash buffer (PBS with 1% FBS and 0.02% NaN3). 100 ul of cell suspension was aliquoted to each FACS tube for staining.

Staining antibodies were purchased from Serotec. Primary antibody: mouse anti-dog CD18 (MCA1780) used at 1:10 dilution. Secondary antibody: goat anti-mouse IgG (H&L) FITC (STAR117 F) used at 1:50 dilution. Negative control antibody: mouse IgG1 FITC/Rat IgG2a RPE (DCO50) used at 1:10 dilution. Cells were incubated with each antibody for 30 min on ice, washed with FACS Wash Buffer and then fixed with 4% Formalin before analysis. Cell populations were analyzed on a FACScan Flow Cytometer (Becton Dickinson) using CellQuest Pro Software.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for improving immunity of a companion animal comprising administering to the companion animal a glucose anti-metabolite in an amount effective to improve the immunity of the companion animal.

2. The method of claim 1 and wherein the glucose anti-metabolite comprises a glucose anti-metabolite component selected from the group consisting of 2-deoxy-D-glucose; 5-thio-D-glucose; 3-O-methylglucose; 1,5-anhydro-D-glucitol; 2,5-anhydro-D-glucitol; 2,5-anhydro-D-mannitol; mannoheptulose; and mixtures and combinations thereof.

3. The method of claim 1 and wherein the companion animal is selected from the group consisting of a canine and a feline.

4. The method of claim 1 and wherein the companion animal is a canine.

5. The method of claim 1 and wherein the companion animal is a feline.

6. The method of claim 1 and wherein the glucose anti-metabolite comprises mannoheptulose.

7. The method of claim 6 and wherein the composition comprises a kibble.

8. The method of claim 7 and wherein the kibble is nutritionally balanced.

9. The method of claim 8 and wherein the kibble comprises less than about 5% mannoheptulose.

10. The method of claim 6 and wherein the administration comprises feeding from about 1 mg to about 50 mg mannoheptulose per kg body weight to the companion animal per day.

11. The method of claim 6 and wherein the administration comprises feeding about 2 mg mannoheptulose per kg body weight to the companion animal per day.

12. The method of claim 6 and wherein administering comprises feeding from about 1 mg to about 1000 mg mannoheptulose per day.

13. The method of claim 6 and wherein administering comprises feeding from about 5 mg to about 100 mg mannoheptulose per day.

14. The method of claim 6 and wherein the composition is a supplement.

15. The method of claim 6 and wherein administering comprises feeding the companion animal a source of mannoheptulose, wherein the source of mannoheptulose is selected from the group consisting of avocado, avocado extract, avocado meal, avocado concentrate, and combinations and mixtures thereof.

16. The method of claim 1 and wherein administering results in improved immunity by way of lower serum 8-OHdG such that DNA damage has been decreased.

17. A method for improving immunity of a companion animal comprising administering to the companion animal a glucose anti-metabolite in an amount effective to improve the immunity of the companion animal, wherein improving the immunity comprises improving the ability of the immune system of the companion animal to respond such that the proliferative ability of T and B immune cells to respond to a stimulation challenge is altered after administration of the glucose anti-metabolite.

18. The method of claim 17 and wherein administering comprises feeding to the companion animal a composition comprising mannoheptulose;
wherein the composition comprises nutritionally balanced kibbles; and
wherein the feeding comprises a daily dosage amount of from about 1 mg/kg to about 50 mg/kg mannoheptulose.

19. A method for improving immunity of a companion animal comprising administering to the companion animal a glucose anti-metabolite in an amount effective to improve the immunity of the companion animal, wherein improving the immunity comprises attenuating the decline of the immune system by attenuating the age associated increase in CD18+ cells.

20. The method of claim 19 and wherein administering comprises feeding to the companion animal a composition comprising mannoheptulose;
wherein the composition comprises nutritionally balanced kibbles; and
wherein the feeding comprises a daily dosage amount of from about 1 mg/kg to about 50 mg/kg mannoheptulose.
